(19) United States
(12) Patent Application Publication
(10) Pub. No.: US 2015/0259709 A1
(43) Pub. Date: Sep. 17, 2015

(54) PROCESSES FOR PRODUCING FLUFF PULP AND ETHANOL FROM SUGARCANE

(71) Applicant: API Intellectual Property Holdings, LLC, Atlanta, GA (US)

(72) Inventors: Theodora RETSINA, Atlanta, GA (US); Vesa PYLKKANEN, Atlanta, GA (US)

(21) Appl. No.: 14/640,255

(22) Filed: Mar. 6, 2015

Related U.S. Application Data
(60) Provisional application No. 61/950,937, filed on Mar. 11, 2014.

(51) Int. Cl. C12P 7/14; D21C 3/20; D21C 3/06; D21H 11/12

(52) U.S. Cl. CPC C12P 7/14; D21H 11/12; D21C 3/20; D21C 3/06

(57) ABSTRACT

The disclosure provides a process for producing fluff pulp and ethanol from sugarcane bagasse or straw, comprising: fractionating the feedstock in the presence of an acid catalyst, a solvent for lignin, and water, to generate a solid/liquid slurry comprising cellulose-rich solids, hemicelluloses, and lignin; separating the solid/liquid slurry into a solid stream and a liquid stream; further treating the cellulose-rich solids to produce fluff pulp; hydrolyzing the hemicelluloses to generate hemicellulose monomers; and fermenting at least a portion of the hemicellulose monomers to cellulosic ethanol. Lignin is removed from the process during one or more steps and combusted to provide energy for process requirements. The process is integrated with, and provides energy to, a first-generation process that ferments sugarcane-derived sucrose to first-generation ethanol. Similar processes are possible with energy cane, corn, and other crops.

### PRIORITY DATA

This patent application is a non-provisional patent application claiming priority to U.S. Provisional Patent App. No. 61/950,937, filed on Mar. 11, 2014, which is hereby incorporated by reference herein.

### FIELD OF THE INVENTION

The present invention generally relates to processes for refining sugarcane and related feedstocks to produce sugars, including sucrose and hemicellulose sugars, which are optionally purified, fermented to biofuels or biochemicals, or recovered for other uses.

### BACKGROUND OF THE INVENTION

Sugarcane is the world's largest crop. Brazil is the largest producer of sugarcane in the world. The world demand for sugar is the primary driver of sugarcane agriculture. Sugarcane accounts for about 80% of sugar produced; most of the rest is made from sugar beets. Sugarcane predominantly grows in the tropical and subtropical regions, and sugar beet predominantly grows in colder temperate regions of the world.

The production of sugar (sucrose) from raw sugarcane is well known. Furthermore, the development of equipment and associated processes for producing sugar from sugarcane stalks has been extensive. Generally, sugar product is produced from a naturally occurring liquid contained within the cells of sugarcane stalks.

In particular, the recovery of sucrose from the cane plant requires the separation of juice from the fibrous material in the structure of the stalk. The tissue inside the rind of the stalk is a matrix of thin-walled parenchyma cells in which are imbedded vascular bundles. Sucrose is present principally in the parenchyma storage cells. These cells are easily ruptured and the most commonly employed methods to extract the juice are by milling or crushing, hot water extraction or “diffusion,” or a combination of these methods. In the diffusion method, cane is typically prepared by knife mills and roller-crusher combinations.

Most sugarcane ethanol mills are in remote locations and use the bagasse to generate the necessary steam and power to run the mill and occasionally export to the local grid. It is therefore not typically viable to divert all the bagasse to the production of cellulosic ethanol. However, the diversion of only the hemicelluloses of the bagasse to the production of cellulosic ethanol is a viable, robust and financially attractive proposition, leading to competitive production of cellulosic ethanol. If a high-value product (such as a specialty pulp) can be produced from the cellulose portion of the bagasse, the economics become attractive.

Such an application can result in incremental cellulosic ethanol production in an existing sugarcane ethanol mill, add revenue with high-value co-products, and still allow the mill to produce (from lignin combustion) its steam and power energy needs.

### SUMMARY OF THE INVENTION

In some variations, the invention provides a process for producing fluff pulp and ethanol from sugarcane bagasse or straw, the process comprising:

(a) obtaining a feedstock comprising sugarcane bagasse and/or straw;

(b) fractionating the feedstock in the presence of an acid catalyst, a solvent for lignin, and water, to generate a solid/liquid slurry comprising cellulose-rich solids, hemicelluloses, and lignin;

(c) separating the solid/liquid slurry into a solid stream comprising the cellulose-rich solids and a liquid stream comprising the hemicelluloses and lignin;

(d) further treating the cellulose-rich solids to produce fluff pulp;

(e) hydrolyzing the hemicelluloses to generate hemicellulose monomers; and

(f) fermenting at least a portion of the hemicellulose monomers to cellulosic ethanol,

wherein the lignin is removed from the process during one or more of steps (b), (c), (d), (e), or (f) and combusted to provide energy; and

wherein the process is integrated with, and provides at least some of the energy to, a first-generation process that ferments sugarcane-derived sucrose to first-generation ethanol.

In some embodiments, the sugarcane-derived sucrose and the sugarcane bagasse and/or straw are each derived from the same starting feedstock. The cellulosic ethanol may be combined with the first-generation ethanol. Utilities and infrastructure may be shared between the process and the first-generation process.

In some embodiments, the acid catalyst is sulfur dioxide and/or the solvent for lignin is ethanol. In some embodiments, step (d) comprises bleaching.

The invention can be applied to other feedstocks. For example, a process for producing fluff pulp and ethanol from corn stover may include the steps of:

(a) obtaining a feedstock comprising corn stover;

(b) fractionating the feedstock in the presence of an acid catalyst, a solvent for lignin, and water, to generate a solid/liquid slurry comprising cellulose-rich solids, hemicelluloses, and lignin;

(c) separating the solid/liquid slurry into a solid stream comprising the cellulose-rich solids and a liquid stream comprising the hemicelluloses and lignin;

(d) further treating the cellulose-rich solids to produce fluff pulp;

(e) hydrolyzing the hemicelluloses to generate hemicellulose monomers; and

(f) fermenting at least a portion of the hemicellulose monomers to cellulosic ethanol,

wherein the lignin is removed from the process during one or more of steps (b), (c), (d), (e), or (f) and combusted to provide energy; and

wherein the process is integrated with, and provides at least some of the energy to, a first-generation process that ferments corn-derived glucose to first-generation ethanol.

In some embodiments, the corn-derived glucose and the corn stover are each derived from the same starting feedstock. The cellulosic ethanol may be combined with the first-generation ethanol. Utilities and infrastructure may be shared between the process and the first-generation process.

Generally, the invention is not limited to fluff pulp. Variations provide a process for producing pulp and ethanol from sugarcane bagasse or straw, the process comprising:

(a) obtaining a feedstock comprising sugarcane bagasse and/or straw;

(b) fractionating the feedstock in the presence of an acid catalyst, a solvent for lignin, and water, to generate a solid/liquid slurry comprising cellulose-rich solids, hemicelluloses, and lignin;

(c) separating the solid/liquid slurry into a solid stream comprising the cellulose-rich solids and a liquid stream comprising the hemicelluloses and lignin;

(d) recovering or further treating the cellulose-rich solids to produce pulp;

(e) hydrolyzing the hemicelluloses to generate hemicellulose monomers; and

(f) fermenting at least a portion of the hemicellulose monomers to cellulosic ethanol,

wherein the lignin is removed from the process during one or more of steps (b), (c), (d), (e), or (f) and combusted to provide energy; and

wherein the process is integrated with, and provides at least some of the energy to, a first-generation process that ferments sugarcane-derived sucrose to first-generation ethanol.

The pulp may be selected from (but not limited to) fluff pulp, market pulp, dissolving pulp, or nanocellulose precursor material.

### BRIEF DESCRIPTION OF THE FIGURE

FIG. 1 is a simplified block-flow diagram depicting the process of some embodiments of the present invention. Dashed lines indicate optional streams.

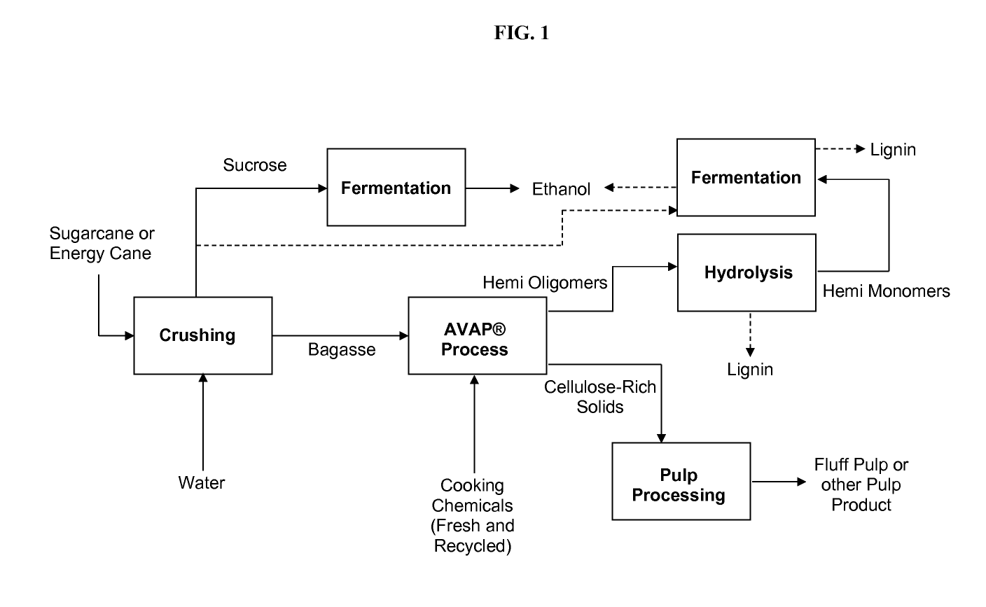

### DETAILED DESCRIPTION OF EMBODIMENTS OF THE INVENTION

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”

Certain exemplary embodiments of the invention will now be described. These embodiments are not intended to limit the scope of the invention as claimed. The order of steps may be varied, some steps may be omitted, and/or other steps may be added. Reference herein to first step, second step, etc. is for illustration purposes only.

In some variations, the invention provides a process for producing fluff pulp and ethanol from sugarcane bagasse or straw, the process comprising:

(a) obtaining a feedstock comprising sugarcane bagasse and/or straw;

(b) fractionating the feedstock in the presence of an acid catalyst, a solvent for lignin, and water, to generate a solid/liquid slurry comprising cellulose-rich solids, hemicelluloses, and lignin;

(c) separating the solid/liquid slurry into a solid stream comprising the cellulose-rich solids and a liquid stream comprising the hemicelluloses and lignin;

(d) further treating the cellulose-rich solids to produce fluff pulp;

(e) hydrolyzing the hemicelluloses to generate hemicellulose monomers; and

(f) fermenting at least a portion of the hemicellulose monomers to cellulosic ethanol,

wherein the lignin is removed from the process during one or more of steps (b), (c), (d),

(e), or (f) and combusted to provide energy; and

wherein the process is integrated with, and provides at least some of the energy to, a first-generation process that ferments sugarcane-derived sucrose to first-generation ethanol.

In some embodiments, the sugarcane-derived sucrose and the sugarcane bagasse and/or straw are each derived from the same starting feedstock. That is, a single source of sugarcane may be processed into sucrose syrup and the bagasse processed according to this disclosure. In other embodiments, bagasse (or other lignocellulosic feedstocks) is processed and integrated with a first-generation ethanol plant, but the lignocellulosic feedstock is not derived from the same starting sugarcane. Note that all references herein to sugarcane are also, in some embodiments, references to energy cane.

The cellulosic ethanol may be combined with the first-generation ethanol, either before, during, or after purification steps. Utilities (e.g., site steam) and infrastructure may be shared between the processes.

In some embodiments, the acid catalyst is sulfur dioxide and/or the solvent for lignin is ethanol. Other acid catalysts may be employed, including sulfur-containing acids, nitrogen-containing acids, phosphorus-containing acids, organic acids, and so on. Other solvents may be employed, as discussed below.

In some embodiments, step (d) comprises bleaching. Any known bleaching sequence may be employed, depending in pulp product requirements.

The invention can be applied to other feedstocks, such as corn, wheat, rice, wild rice, millet, sorghum, barley, oats, rye, teff, and others.

For example, a process for producing fluff pulp and ethanol from corn stover may include the steps of:

(a) obtaining a feedstock comprising corn stover;

(b) fractionating the feedstock in the presence of an acid catalyst, a solvent for lignin, and water, to generate a solid/liquid slurry comprising cellulose-rich solids, hemicelluloses, and lignin;

(c) separating the solid/liquid slurry into a solid stream comprising the cellulose-rich solids and a liquid stream comprising the hemicelluloses and lignin;

(d) further treating the cellulose-rich solids to produce fluff pulp;

(e) hydrolyzing the hemicelluloses to generate hemicellulose monomers; and

(f) fermenting at least a portion of the hemicellulose monomers to cellulosic ethanol,

wherein the lignin is removed from the process during one or more of steps (b), (c), (d),

(e), or (f) and combusted to provide energy; and

wherein the process is integrated with, and provides at least some of the energy to, a first-generation process that ferments corn-derived glucose to first-generation ethanol.

In some embodiments, the corn-derived glucose and the corn stover are each derived from the same starting feedstock. The cellulosic ethanol may be combined with the first-generation ethanol. Utilities and infrastructure may be shared between the process and the first-generation process.

Generally, the invention is not limited to fluff pulp. Variations provide a process for producing pulp and ethanol from sugarcane bagasse or straw, the process comprising:

(a) obtaining a feedstock comprising sugarcane bagasse and/or straw;

(b) fractionating the feedstock in the presence of an acid catalyst, a solvent for lignin, and water, to generate a solid/liquid slurry comprising cellulose-rich solids, hemicelluloses, and lignin;

(c) separating the solid/liquid slurry into a solid stream comprising the cellulose-rich solids and a liquid stream comprising the hemicelluloses and lignin;

(d) recovering or further treating the cellulose-rich solids to produce pulp;

(e) hydrolyzing the hemicelluloses to generate hemicellulose monomers; and

(f) fermenting at least a portion of the hemicellulose monomers to cellulosic ethanol,

wherein the lignin is removed from the process during one or more of steps (b), (c), (d),

(e), or (f) and combusted to provide energy; and

wherein the process is integrated with, and provides at least some of the energy to, a first-generation process that ferments sugarcane-derived sucrose to first-generation ethanol.

The pulp may be selected from (but not limited to) fluff pulp, market pulp, dissolving pulp, or nanocellulose precursor material.

In certain embodiments, the feedstock is sugarcane and the lignocellulosic material contains sugarcane bagasse and optionally sugarcane straw. That is, bagasse may be combined with sugarcane straw (also known as trash). The sugarcane straw may be subjected to extracting with steam and/or hot water to produce cellulose-rich solids and an extract liquor containing hemicellulosic oligomers. This step could be done in isolation from the extraction of bagasse (containing sucrose) or it could be done in combination, where the bagasse and straw are co-fed.

In some embodiments, the bagasse and straw are not separated in the first place. In many sugar mills around the world, burning the standing sugarcane to facilitate cutting and lifting for transport to the mill is common practice. When standing sugarcane is not burned, all of the lignocellulosic material may be processed by the methods disclosed herein.

Some amount of sucrose may be intentionally left in the lignocellulosic material. The extracted lignocellulosic material may retains at least 2%, 3%, 4%, 5%, or more of the initial sucrose contained in the feedstock, in various embodiments. In some embodiments, the extracted lignocellulosic material retains from about 1% to about 10% of the initial sucrose contained in the feedstock, such as about 2% to about 7% or about 3% to about 6% of the initial sucrose contained in the feedstock. In some embodiments, the extracted lignocellulosic material retains more than about 10%, such as about 20%, 30%, 40%, 50% or more (but less than all) of the initial sucrose contained in the feedstock.

Mechanically treating the feedstock to generate a sucrose-rich stream and sucrose-depleted lignocellulosic material is well-known in the art of sugarcane processing. The reference here to mechanical treatment is for the extraction of cane juice, not the initial mechanical harvesting of sugarcane (e.g., cutting and lifting), although there can be some overlap of functions.

In some embodiments, the cut sugarcane stalks are initially transferred onto a conveyer table where they are subjected to a standard washing step to reduce impurities on the surface of the stalks. Subsequently, the sugarcane stalks may be conveyed through a standard crushing or chopping apparatus to reduce the stalks into smaller individual pieces for feeding through a series of roller mills, as is well-known to those skilled in the art.

It may be preferable to limit the hydraulic pressure during crushing or chopping so that some sucrose is retained in the bagasse, if desired. The limited hydraulic pressure may also minimize the undesirable extraction of natural waxes, ferrous compounds and other minerals from the cortex of the sugarcane.

In some embodiments, hot water maceration may aid in the extraction of sucrose. Hot water tends to dissolve natural waxes and minerals in the hard, outer cortex of the cane stalk. It may be desirable to limit the temperature and/or residence time of hot water maceration so that some sucrose is retained in the bagasse.

In some embodiments, step (e) employs an acid catalyst for hydrolyzing the hemicellulosic oligomers. In other embodiments, step (e) employs an enzyme catalyst for hydrolyzing the hemicellulosic oligomers. Step (e) may also utilize lignosulfonic acids generated during the initial fractionation in step (b).

The process may include treatment of the extract liquor containing hemicellulosic oligomers to form a hydrolysate comprising fermentable hemicellulose sugars. In some embodiments, the biomass extract is hydrolyzed using dilute acidic conditions at temperatures between about 100° C. and 190° C., for example about 120° C., 130° C., 140° C., 150° C., 160° C., or 170° C., and preferably from 120° C. to 150° C. The sucrose may be hydrolyzed to glucose and fructose, at least to some extent.

The acid may be selected from sulfuric acid, sulfurous acid, or sulfur dioxide. Alternatively, or additionally, the acid may include formic acid, acetic acid, or oxalic acid from the cooking liquor or recycled from previous hydrolysis. Alternatively, hemicellulase enzymes may be used instead of acid hydrolysis. The lignin from this step may be separated and recovered, or sent directly to a boiler.

Various moisture levels may be associated with the starting biomass. The biomass feedstock need not be, but may be, relatively dry. In general, the biomass is in the form of a particulate or chip, but particle size is not critical in this invention.

The solvent for lignin preferably facilitates a higher mass transfer rate of the sulfur dioxide into the lignocellulosic biomass, compared to the mass transfer rate of sulfur dioxide into the lignocellulosic biomass with water alone. For example, ethanol facilitates better SO₂ mass transfer because ethanol (with dissolved SO₂) is able to penetrate into biomass pores more efficiently than water.

In some embodiments, the solvent for lignin comprises an oxygenated hydrocarbon, such as an aliphatic alcohol which may be a C₁-C₈ alcohol, for example, or an aromatic alcohol, such as phenol. In some embodiments, the solvent for lignin comprises an aliphatic or aromatic hydrocarbon.

In some embodiments, the solvent for lignin comprises an organic acid. For example, without limitation, the organic acid may be selected from the group consisting of acetic acid, formic acid, oxalic acid, lactic acid, propionic acid, 3-hydroxypropionic acid, malonic acid, aspartic acid, fumaric acid, malic acid, succinic acid, glutaric acid, adipic acid, citric acid, itaconic acid, levulinic acid, ascorbic acid, gluconic acid, kojic acid, and combinations thereof

In these or other embodiments, the solvent for lignin comprises an inorganic acid, such as concentrated phosphoric acid. In certain embodiments, the solvent for lignin comprises an ionic liquid.

The process may further include recovering the lignin, lignosulfonates, or both of these. Recovery of lignin typically involves removal of solvent, dilution with water, adjustment of temperature or pH, addition of an acid or base, or some combination thereof

The sulfur dioxide may be present in a liquid-phase concentration of about 1 wt % to about 50 wt % during step (a), or about 6 wt % to about 30 wt %, or about 9 wt % to about 20 wt %, in various embodiments.

Step (b) typically includes washing of the cellulose-rich solids, which preferably includes countercurrent washing of the cellulose-rich solids.

Hydrolyzing the hemicellulose contained in the liquor, in step (c), may be catalyzed by lignosulfonic acids that are generated during step (a).

The fermentation product may include an organic acid, such as (but not limited to) organic acids selected from the group consisting of formic acid, acetic acid, oxalic acid, lactic acid, propionic acid, 3-hydroxypropionic acid, malonic acid, aspartic acid, fumaric acid, malic acid, succinic acid, glutaric acid, adipic acid, citric acid, itaconic acid, levulinic acid, ascorbic acid, gluconic acid, kojic acid, threonine, glutamic acid, proline, lysine, alanine, serine, and any isomers, derivatives, or combinations thereof. In certain embodiments, the organic acid is succinic acid. “Derivatives” may be salts of these acids, or esters, or reaction products to convert the acid to another molecule that is not an acid. For example, when the fermentation product is succinic acid, it may be further converted to 1,4-butanediol as a derivative using known hydrotreating chemistry.

The fermentation product may include an oxygenated compound, such as (but not limited to) oxygenated compounds selected from the group consisting of ethanol, propanol, butanol, pentanol, hexanol, heptanol, octanol, glycerol, sorbitol, propanediol, butanediol, butanetriol, pentanediol, hexanediol, acetone, acetoin, butyrolactone, 3-hydroxybutyrolactone, and any isomers, derivatives, or combinations thereof

In some embodiments, the oxygenated compound is a C₃ or higher alcohol or diol, such as 1-butanol, isobutanol, 1,4-butanediol, 2,3-butanediol, or mixtures thereof

The fermentation product may include a hydrocarbon, such as isoprene, farnasene, and related compounds.

Multiple fermentation products may be produced in a single fermentor, in co-product production or as a result of byproducts due to contaminant microorganisms. For example, during fermentation to produce lactic acid, ethanol is a common byproduct due to contamination (and vice-versa).

Multiple fermentation products may be produced in separate fermentors. In some embodiments, a first fermentation product, such as an organic acid, is produced from glucose (hydrolyzed cellulose) while a second fermentation product, such as ethanol, is produced from hemicellulose sugars. Or, in some embodiments, different fermentations are directed to portions of feedstock having varying particle size, crystallinity, or other properties.

In some embodiments, different fermentations are directed to portions of whole biomass that is separated into a starch or sucrose-rich fraction, and a cellulose-rich fraction (for example, corn starch/stover or sugarcane syrup/bagasse). For example, from raw corn, an organic acid or polyol may be produced from starch (hydrolyzed to glucose), the same or a different organic acid or polyol may be produced from cellulose (hydrolyzed to glucose), and ethanol may be produced from hemicellulose sugars. Many variations are possible, as will be recognized by a person skilled in the biorefinery art, in view of the present disclosure.

The solvent for lignin may include a component that is the same as the fermentation product. In some embodiments, the solvent for lignin is the same compound as the fermentation product. For example, the solvent and the fermentation product may be 1-butanol, or lactic acid, succinic acid, or 1,4-butanediol. Of course, other solvents may be present even when these products are utilized as solvents or co-solvents. Beneficially, a portion of the fermentation product may be recycled to step (a) for use as the solvent for lignin.

In some embodiments, the fermentation product includes an enzymatically isomerized variant of at least a portion of the fermentable sugars. For example, the enzymatically isomerized variant may include fructose which is isomerized from glucose. In some embodiments, glucose, which is normally D-glucose, is isomerized with enzymes to produce L-glucose.

In some embodiments, the fermentation product includes one or more proteins, amino acids, enzymes, or microorganisms. Such fermentation products may be recovered and used within the process; for example, cellulase or hemicellulase enzymes may be used for hydrolyzing cellulose-rich solids or hemicellulose oligomers.

In some embodiments, the hydrolysis catalyst is present in a liquid-phase concentration of about 1 wt % to about 50 wt % during step (a), such as about 6 wt % to about 30 wt %, or about 9 wt % to about 20 wt %. The hydrolysis catalyst in step (a) may be selected from the group consisting of sulfur dioxide, sulfur trioxide, sulfurous acid, sulfuric acid, sulfonic acid, lignosulfonic acid, elemental sulfur, polysulfides, and combinations or derivatives thereof.

In some embodiments, the hydrolyzing in step (c) utilizes the hydrolysis catalyst from step (a), or a reaction product thereof. For example, in certain embodiments the hydrolysis catalyst is sulfur dioxide and the reaction product is lignosulfonic acid. In other embodiments, the hydrolyzing in step (c) utilizes hemicellulase enzymes as hydrolysis catalyst.

In some embodiments, the solvent for lignin also contains the functionality of a hydrolysis catalyst, i.e. there is not a separate hydrolysis catalyst present. In particular, when the solvent for lignin is an organic acid, it may also function as the hydrolysis catalyst.

In some embodiments, the process further comprises saccharifying at least some of the cellulose-rich solids to produce glucose. In these or other embodiments, the process further comprises recovering or further treating or reacting at least some of the cellulose-rich solids as a pulp precursor or product. When glucose is produced (by acid or enzyme hydrolysis of the cellulose), that glucose may form part of the fermentable sugars, either separately from the hemicellulose-derived fermentable sugars, or as a combined sugar stream.

In some embodiments, the fermentation product is ethanol, 1-butanol, succinic acid, 1,4-butanediol, or a combination thereof. In some embodiments, the solvent for lignin includes a component that is the same as the fermentation product, or is the same compound as the fermentation product. Thus a portion of the fermentation product may be recycled for use as the solvent for lignin.

At least a portion of the hemicellulose sugar stream may be fermented to a fermentation product, such as ethanol. In these or other embodiments, at least a portion of the hemicellulose sugar stream may be recovered as purified hemicellulose sugars.

At least a portion of the sucrose-rich stream may be fermented to ethanol or another fermentation product. In these or other embodiments, at least a portion of the sucrose-rich stream may be recovered as purified sucrose sugar. In a certain embodiment, the sucrose is purified and sold as a sugar product while the hemicellulose sugars are fermented to cellulosic ethanol or another fermentation product.

In some embodiments, the fermentable hemicellulose sugars are recovered from solution, in purified form. In some embodiments, the fermentable hemicellulose sugars are fermented to produce of biochemicals or biofuels such as (but by no means limited to) ethanol, 1-butanol, isobutanol, acetic acid, lactic acid, succinic acid, or any other fermentation products. A purified fermentation product may be produced by distilling the fermentation product, which will also generate a distillation bottoms stream containing residual solids. A bottoms evaporation stage may be used, to produce residual solids.

Following fermentation, residual solids (such as distillation bottoms) may be recovered, or burned in solid or slurry form, or recycled. Use of the fermentation residual solids may require further removal of minerals. Generally, any leftover solids may be used for burning as additional liquefied biomass, after concentration of the distillation bottoms.

Part or all of the residual solids may be co-combusted, if desired. Alternatively, or additionally, the process may include recovering the residual solids as a fermentation co-product in solid, liquid, or slurry form. The fermentation co-product may be used as a fertilizer or fertilizer component, since it will typically be rich in potassium, nitrogen, and/or phosphorous.

There are many options that may be employed regarding sugar recovery or further processing. In some embodiments, the sucrose-rich stream is recovered and purified as a sugar product in dry form. The sucrose that is diverted to the hemicellulose sugar stream, according to the processes disclosed, may be recovered in principle but typically will be converted by fermentation into a product such as ethanol.

It is possible to convert the main sucrose fraction into a product, such as a biochemical (e.g., lactic acid) while the hemicellulose sugars are converted to a different product, such as a biofuel (e.g., ethanol). It is also possible to recombine some or all of the sugars, if desired, for common fermentation or other processing.

In addition, all of these options are dynamic. A plant may operate to maximize pure sugar for some period of time and then, depending on economic conditions, time of year, weather factors, or policy changes, shift to production of biofuels/biochemicals from the sucrose. Along with these dynamic adjustments in operation, the amount of residual sucrose remaining in the bagasse according to the disclosed process may vary, to shift the product portfolio. For example if the balance will shift from sugars toward biofuels/biochemicals, it may be beneficial to allow more sucrose to remain in the bagasse to take advantage of fermentation capacity, etc.

Reaction conditions and operation sequences may vary widely. Some embodiments employ conditions described in U.S. Pat. No. 8,030,039, issued Oct. 4, 2011; U.S. Pat. No. 8,038,842, issued Oct. 11, 2011; U.S. Pat. No. 8,268,125, issued Sep. 18, 2012; and U.S. patent application Ser. Nos. 13/004,431; 12/234,286; 13/585,710; 12/250,734; 12/397,284; 12/304,046; 13/500,916; 13/626,220; 12/854,869; 61/732,047; 61/735,738; 61/739,343; 61/747,010; 61/747,105; 61/747,376; 61/747,379; 61/747,382; and/or 61/747,408, including the prosecution histories thereof. Each of these commonly owned patent applications is hereby incorporated by reference herein in its entirety. In some embodiments, the process is a variation of the AVAP® process technology which is commonly owned with the assignee of this patent application.

In some embodiments, a first process step is “cooking” (equivalently, “digesting”) which fractionates the three lignocellulosic material components (cellulose, hemicellulose, and lignin) to allow easy downstream removal. Specifically, hemicelluloses are dissolved and over 50% are completely hydrolyzed; cellulose is separated but remains resistant to hydrolysis; and part of the lignin is sulfonated into water-soluble lignosulfonates.

The lignocellulosic material is processed in a solution (cooking liquor) of aliphatic alcohol, water, and sulfur dioxide. The cooking liquor preferably contains at least 10 wt %, such as at least 20 wt %, 30 wt %, 40 wt %, or 50 wt % of a solvent for lignin. For example, the cooking liquor may contain about 30-70 wt % solvent, such as about 50 wt % solvent. The solvent for lignin may be an aliphatic alcohol, such as methanol, ethanol, 1-propanol, 2-propanol, 1-butanol, 2-butanol, isobutanol, 1-pentanol, 1-hexanol, or cyclohexanol. The solvent for lignin may be an aromatic alcohol, such as phenol or cresol. Other lignin solvents are possible, such as (but not limited to) glycerol, methyl ethyl ketone, or diethyl ether. Combinations of more than one solvent may be employed.

Preferably, enough solvent is included in the extractant mixture to dissolve the lignin present in the starting material. The solvent for lignin may be completely miscible, partially miscible, or immiscible with water, so that there may be more than one liquid phase. Potential process advantages arise when the solvent is miscible with water, and also when the solvent is immiscible with water. When the solvent is water-miscible, a single liquid phase forms, so mass transfer of lignin and hemicellulose extraction is enhanced, and the downstream process must only deal with one liquid stream. When the solvent is immiscible in water, the extractant mixture readily separates to form liquid phases, so a distinct separation step can be avoided or simplified. This can be advantageous if one liquid phase contains most of the lignin and the other contains most of the hemicellulose sugars, as this facilitates recovering the lignin from the hemicellulose sugars.

The cooking liquor preferably contains sulfur dioxide and/or sulfurous acid (H₂SO₃). The cooking liquor preferably contains SO₂, in dissolved or reacted form, in a concentration of at least 3 wt %, preferably at least 6 wt %, more preferably at least 8 wt %, such as about 9 wt %, 10 wt %, 11 wt %, 12 wt %, 13 wt %, 14 wt %, 15 wt %, 20 wt %, 25 wt %, 30 wt % or higher. The cooking liquor may also contain one or more species, separately from SO₂, to adjust the pH. The pH of the cooking liquor is typically about 4 or less.

Sulfur dioxide is a preferred acid catalyst, because it can be recovered easily from solution after hydrolysis. The majority of the SO₂ from the hydrolysate may be stripped and recycled back to the reactor. Recovery and recycling translates to less lime required compared to neutralization of comparable sulfuric acid, less solids to dispose of, and less separation equipment. The increased efficiency owing to the inherent properties of sulfur dioxide mean that less total acid or other catalysts may be required. This has cost advantages, since sulfuric acid can be expensive. Additionally, and quite significantly, less acid usage also will translate into lower costs for a base (e.g., lime) to increase the pH following hydrolysis, for downstream operations. Furthermore, less acid and less base will also mean substantially less generation of waste salts (e.g., gypsum) that may otherwise require disposal.

In some embodiments, an additive may be included in amounts of about 0.1 wt % to 10 wt % or more to increase cellulose viscosity. Exemplary additives include ammonia, ammonia hydroxide, urea, anthraquinone, magnesium oxide, magnesium hydroxide, sodium hydroxide, and their derivatives.

The cooking is performed in one or more stages using batch or continuous digestors. Solid and liquid may flow cocurrently or countercurrently, or in any other flow pattern that achieves the desired fractionation. The cooking reactor may be internally agitated, if desired.

Depending on the lignocellulosic material to be processed, the cooking conditions are varied, with temperatures from about 65° C. to 175° C., for example 75° C., 85° C., 95° C., 105° C., 115° C., 125° C., 130° C., 135° C., 140° C., 145° C., 150° C., 155° C., 165° C. or 170° C., and corresponding pressures from about 1 atmosphere to about 15 atmospheres in the liquid or vapor phase. The cooking time of one or more stages may be selected from about 15 minutes to about 720 minutes, such as about 30, 45, 60, 90, 120, 140, 160, 180, 250, 300, 360, 450, 550, 600, or 700 minutes. Generally, there is an inverse relationship between the temperature used during the digestion step and the time needed to obtain good fractionation of the biomass into its constituent parts.

The cooking liquor to lignocellulosic material ratio may be selected from about 1 to about 10, such as about 2, 3, 4, 5, or 6. In some embodiments, biomass is digested in a pressurized vessel with low liquor volume (low ratio of cooking liquor to lignocellulosic material), so that the cooking space is filled with ethanol and sulfur dioxide vapor in equilibrium with moisture. The cooked biomass is washed in alcohol-rich solution to recover lignin and dissolved hemicelluloses, while the remaining pulp is further processed. In some embodiments, the process of fractionating lignocellulosic material comprises vapor-phase cooking of lignocellulosic material with aliphatic alcohol (or other solvent for lignin), water, and sulfur dioxide. See, for example, U.S. Pat. Nos. 8,038,842 and 8,268,125 which are incorporated by reference herein.

A portion or all of the sulfur dioxide may be present as sulfurous acid in the extract liquor. In certain embodiments, sulfur dioxide is generated in situ by introducing sulfurous acid, sulfite ions, bisulfite ions, combinations thereof, or a salt of any of the foregoing. Excess sulfur dioxide, following hydrolysis, may be recovered and reused.

In some embodiments, sulfur dioxide is saturated in water (or aqueous solution, optionally with an alcohol) at a first temperature, and the hydrolysis is then carried out at a second, generally higher, temperature. In some embodiments, sulfur dioxide is sub-saturated. In some embodiments, sulfur dioxide is super-saturated. In some embodiments, sulfur dioxide concentration is selected to achieve a certain degree of lignin sulfonation, such as 1%, 2%, 3%, 4%, 5%, 6%, 7%, 8%, 9%, or 10% sulfur content. SO₂ reacts chemically with lignin to form stable lignosulfonic acids which may be present both in the solid and liquid phases.

The concentration of sulfur dioxide, additives, and aliphatic alcohol (or other solvent) in the solution and the time of cook may be varied to control the yield of cellulose and hemicellulose in the pulp. The concentration of sulfur dioxide and the time of cook may be varied to control the yield of lignin versus lignosulfonates in the hydrolysate. In some embodiments, the concentration of sulfur dioxide, temperature, and the time of cook may be varied to control the yield of fermentable sugars.

Once the desired amount of fractionation of both hemicellulose and lignin from the solid phase is achieved, the liquid and solid phases are separated. Conditions for the separation may be selected to minimize the reprecipitation of the extracted lignin on the solid phase. This is favored by conducting separation or washing at a temperature of at least the glass-transition temperature of lignin (about 120° C.).

The physical separation can be accomplished either by transferring the entire mixture to a device that can carry out the separation and washing, or by removing only one of the phases from the reactor while keeping the other phase in place. The solid phase can be physically retained by appropriately sized screens through which liquid can pass. The solid is retained on the screens and can be kept there for successive solid-wash cycles. Alternately, the liquid may be retained and solid phase forced out of the reaction zone, with centrifugal or other forces that can effectively transfer the solids out of the slurry. In a continuous system, countercurrent flow of solids and liquid can accomplish the physical separation.

The recovered solids normally will contain a quantity of lignin and sugars, some of which can be removed easily by washing. The washing-liquid composition can be the same as or different than the liquor composition used during fractionation. Multiple washes may be performed to increase effectiveness. Preferably, one or more washes are performed with a composition including a solvent for lignin, to remove additional lignin from the solids, followed by one or more washes with water to displace residual solvent and sugars from the solids. Recycle streams, such as from solvent-recovery operations, may be used to wash the solids.

After separation and washing as described, a solid phase and at least one liquid phase are obtained. The solid phase contains substantially undigested cellulose. A single liquid phase is usually obtained when the solvent and the water are miscible in the relative proportions that are present. In that case, the liquid phase contains, in dissolved form, most of the lignin originally in the starting lignocellulosic material, as well as soluble monomeric and oligomeric sugars formed in the hydrolysis of any hemicellulose that may have been present. Multiple liquid phases tend to form when the solvent and water are wholly or partially immiscible. The lignin tends to be contained in the liquid phase that contains most of the solvent. Hemicellulose hydrolysis products tend to be present in the liquid phase that contains most of the water.

In some embodiments, hydrolysate from the cooking step is subjected to pressure reduction. Pressure reduction may be done at the end of a cook in a batch digestor, or in an external flash tank after extraction from a continuous digestor, for example. The flash vapor from the pressure reduction may be collected into a cooking liquor make-up vessel. The flash vapor contains substantially all the unreacted sulfur dioxide which may be directly dissolved into new cooking liquor. The cellulose is then removed to be washed and further treated as desired.

A process washing step recovers the hydrolysate from the cellulose. The washed cellulose is pulp that may be used for various purposes (e.g., paper or nanocellulose production). The weak hydrolysate from the washer continues to the final reaction step; in a continuous digestor this weak hydrolysate may be combined with the extracted hydrolysate from the external flash tank. In some embodiments, washing and/or separation of hydrolysate and cellulose-rich solids is conducted at a temperature of at least about 100° C., 110° C., or 120° C. The washed cellulose may also be used for glucose production via cellulose hydrolysis with enzymes or acids.

In another reaction step, the hydrolysate may be further treated in one or multiple steps to hydrolyze the oligomers into monomers. This step may be conducted before, during, or after the removal of solvent and sulfur dioxide. The solution may or may not contain residual solvent (e.g. alcohol). In some embodiments, sulfur dioxide is added or allowed to pass through to this step, to assist hydrolysis. In these or other embodiments, an acid such as sulfurous acid or sulfuric acid is introduced to assist with hydrolysis. In some embodiments, the hydrolysate is autohydrolyzed by heating under pressure. In some embodiments, no additional acid is introduced, but lignosulfonic acids produced during the initial cooking are effective to catalyze hydrolysis of hemicellulose oligomers to monomers. In various embodiments, this step utilizes sulfur dioxide, sulfurous acid, sulfuric acid at a concentration of about 0.01 wt % to 30 wt %, such as about 0.05 wt %, 0.1 wt %, 0.2 wt %, 0.5 wt %, 1 wt %, 2 wt %, 5 wt %, 10 wt %, or 20 wt %. This step may be carried out at a temperature from about 100° C. to 220° C., such as about 110° C., 120° C., 130° C., 140° C., 150° C., 160° C., 170° C., 180° C., 190° C., 200° C., or 210° C. Heating may be direct or indirect to reach the selected temperature.

The reaction step produces fermentable sugars which can then be concentrated by evaporation to a fermentation feedstock. Concentration by evaporation may be accomplished before, during, or after the treatment to hydrolyze oligomers. The final reaction step may optionally be followed by steam stripping of the resulting hydrolysate to remove and recover sulfur dioxide and alcohol, and for removal of potential fermentation-inhibiting side products. The evaporation process may be under vacuum or pressure, from about −0.1 atmospheres to about 10 atmospheres, such as about 0.1 atm, 0.3 atm, 0.5 atm, 1.0 atm, 1.5 atm, 2 atm, 4 atm, 6 atm, or 8 atm.

Recovering and recycling the sulfur dioxide may utilize separations such as, but not limited to, vapor-liquid disengagement (e.g. flashing), steam stripping, extraction, or combinations or multiple stages thereof. Various recycle ratios may be practiced, such as about 0.1, 0.2, 0.3, 0.4, 0.5, 0.6, 0.7, 0.8, 0.9, 0.95, or more. In some embodiments, about 90-99% of initially charged SO₂ is readily recovered by distillation from the liquid phase, with the remaining 1-10% (e.g., about 3-5%) of the SO₂ primarily bound to dissolved lignin in the form of lignosulfonates.

In a preferred embodiment, the evaporation step utilizes an integrated alcohol stripper and evaporator. Evaporated vapor streams may be segregated so as to have different concentrations of organic compounds in different streams. Evaporator condensate streams may be segregated so as to have different concentrations of organic compounds in different streams. Alcohol may be recovered from the evaporation process by condensing the exhaust vapor and returning to the cooking liquor make-up vessel in the cooking step. Clean condensate from the evaporation process may be used in the washing step.

In some embodiments, an integrated alcohol stripper and evaporator system is employed, wherein aliphatic alcohol is removed by vapor stripping, the resulting stripper product stream is concentrated by evaporating water from the stream, and evaporated vapor is compressed using vapor compression and is reused to provide thermal energy.

The hydrolysate from the evaporation and final reaction step contains mainly fermentable sugars but may also contain lignin depending on the location of lignin separation in the overall process configuration. The hydrolysate may be concentrated to a concentration of about 5 wt % to about 60 wt % solids, such as about 10 wt %, 15 wt %, 20 wt %, 25 wt %, 30 wt %, 35 wt %, 40 wt %, 45 wt %, 50 wt % or 55 wt % solids. The hydrolysate contains fermentable sugars.

The process lignin separation step is for the separation of lignin from the hydrolysate and can be located before or after the final reaction step and evaporation. If located after, then lignin will precipitate from the hydrolysate since alcohol has been removed in the evaporation step. The remaining water-soluble lignosulfonates may be precipitated by converting the hydrolysate to an alkaline condition (pH higher than 7) using, for example, an alkaline earth oxide, preferably calcium oxide (lime). The combined lignin and lignosulfonate precipitate may be filtered. The lignin and lignosulfonate filter cake may be dried as a co-product or burned or gasified for energy production. The hydrolysate from filtering may be recovered and sold as a concentrated sugar solution product or further processed in a subsequent fermentation or other reaction step.

Native (non-sulfonated) lignin is hydrophobic, while lignosulfonates are hydrophilic. Hydrophilic lignosulfonates may have less propensity to clump, agglomerate, and stick to surfaces. Even lignosulfonates that do undergo some condensation and increase of molecular weight, will still have an HSO₃ group that will contribute some solubility (hydrophilic).

In some embodiments, the soluble lignin precipitates from the hydrolysate after solvent has been removed in the evaporation step. In some embodiments, reactive lignosulfonates are selectively precipitated from hydrolysate using excess lime (or other base, such as ammonia) in the presence of aliphatic alcohol. In some embodiments, hydrated lime is used to precipitate lignosulfonates. In some embodiments, part of the lignin is precipitated in reactive form and the remaining lignin is sulfonated in water-soluble form.

A lignin product can be readily obtained from a liquid phase using one or more of several methods. One simple technique is to evaporate off all liquid, resulting in a solid lignin-rich residue. This technique would be especially advantageous if the solvent for lignin is water-immiscible. Another method is to cause the lignin to precipitate out of solution. Some of the ways to precipitate the lignin include (1) removing the solvent for lignin from the liquid phase, but not the water, such as by selectively evaporating the solvent from the liquid phase until the lignin is no longer soluble; (2) diluting the liquid phase with water until the lignin is no longer soluble; and (3) adjusting the temperature and/or pH of the liquid phase. Methods such as centrifugation can then be utilized to capture the lignin. Yet another technique for removing the lignin is continuous liquid-liquid extraction to selectively remove the lignin from the liquid phase, followed by removal of the extraction solvent to recover relatively pure lignin.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A process for producing fluff pulp and ethanol from sugarcane bagasse or straw, said process comprising:
(a) obtaining a feedstock comprising sugarcane bagasse and/or straw;
(b) fractionating said feedstock in the presence of an acid catalyst, a solvent for lignin, and water, to generate a solid/liquid slurry comprising cellulose-rich solids, hemicelluloses, and lignin;
(c) separating said solid/liquid slurry into a solid stream comprising said cellulose-rich solids and a liquid stream comprising said hemicelluloses and lignin;
(d) further treating said cellulose-rich solids to produce fluff pulp;
(e) hydrolyzing said hemicelluloses to generate hemicellulose monomers; and
(f) fermenting at least a portion of said hemicellulose monomers to cellulosic ethanol,
wherein said lignin is removed from said process during one or more of steps (b), (c), (d),
(e), or (f) and combusted to provide energy; and
wherein said process is integrated with, and provides at least some of said energy to, a first-generation process that ferments sugarcane-derived sucrose to first-generation ethanol.

2. The process of claim 1, wherein said sugarcane-derived sucrose and said sugarcane bagasse and/or straw are each derived from the same starting feedstock.

3. The process of claim 1, wherein said cellulosic ethanol is combined with said first-generation ethanol.

4. The process of claim 1, wherein utilities and infrastructure are shared between said process and said first-generation process.

5. The process of claim 1, wherein said acid catalyst is sulfur dioxide.

6. The process of claim 1, wherein said solvent for lignin is ethanol.

7. The process of claim 1, wherein step (d) comprises bleaching.

8. A process for producing fluff pulp and ethanol from corn stover, said process comprising:
(a) obtaining a feedstock comprising corn stover;
(b) fractionating said feedstock in the presence of an acid catalyst, a solvent for lignin, and water, to generate a solid/liquid slurry comprising cellulose-rich solids, hemicelluloses, and lignin;
(c) separating said solid/liquid slurry into a solid stream comprising said cellulose-rich solids and a liquid stream comprising said hemicelluloses and lignin;
(d) further treating said cellulose-rich solids to produce fluff pulp;
(e) hydrolyzing said hemicelluloses to generate hemicellulose monomers; and
(f) fermenting at least a portion of said hemicellulose monomers to cellulosic ethanol,
wherein said lignin is removed from said process during one or more of steps (b), (c), (d),
(e), or (f) and combusted to provide energy; and
wherein said process is integrated with, and provides at least some of said energy to, a first-generation process that ferments corn-derived glucose to first-generation ethanol.

9. The process of claim 8, wherein said corn-derived glucose and said corn stover are each derived from the same starting feedstock.

10. The process of claim 8, wherein said cellulosic ethanol is combined with said first-generation ethanol.

11. The process of claim 8, wherein utilities and infrastructure are shared between said process and said first-generation process.

12. The process of claim 8, wherein said acid catalyst is sulfur dioxide.

13. The process of claim 8, wherein said solvent for lignin is ethanol.

14. The process of claim 8, wherein step (d) comprises bleaching.

15. A process for producing pulp and ethanol from sugarcane bagasse or straw, said process comprising:
(a) obtaining a feedstock comprising sugarcane bagasse and/or straw;
(b) fractionating said feedstock in the presence of an acid catalyst, a solvent for lignin, and water, to generate a solid/liquid slurry comprising cellulose-rich solids, hemicelluloses, and lignin;
(c) separating said solid/liquid slurry into a solid stream comprising said cellulose-rich solids and a liquid stream comprising said hemicelluloses and lignin;
(d) recovering or further treating said cellulose-rich solids to produce pulp;
(e) hydrolyzing said hemicelluloses to generate hemicellulose monomers; and
(f) fermenting at least a portion of said hemicellulose monomers to cellulosic ethanol,
wherein said lignin is removed from said process during one or more of steps (b), (c), (d),
(e), or (f) and combusted to provide energy; and
wherein said process is integrated with, and provides at least some of said energy to, a first-generation process that ferments sugarcane-derived sucrose to first-generation ethanol.

16. The process of claim 15, wherein said sugarcane-derived sucrose and said sugarcane bagasse and/or straw are each derived from the same starting feedstock.

17. The process of claim 15, wherein said cellulosic ethanol is combined with said first-generation ethanol.

18. The process of claim 15, wherein utilities and infrastructure are shared between said process and said first-generation process.

19. The process of claim 15, wherein said acid catalyst is sulfur dioxide.

20. The process of claim 15, wherein said solvent for lignin is ethanol.

21. The process of claim 15, wherein step (d) comprises bleaching.

22. The process of claim 15, wherein said pulp is fluff pulp.

23. The process of claim 15, wherein said pulp is market pulp.

24. The process of claim 15, wherein said pulp is dissolving pulp.

25. The process of claim 15, wherein said pulp is a nanocellulose precursor.
